(19) United States
(12) Patent Application Publication
(10) Pub. No.: US 2025/0093294 A1
(43) Pub. Date: Mar. 20, 2025

(54) WATER QUALITY MONITORING METHOD AND DEVICE

(71) Applicant: UNIVERSITY OF NEWCASTLE UPON TYNE, Newcastle upon Tyne, Tyne and Wear (GB)

(72) Inventors: Martin Spurr, Newcastle upon Tyne (GB); Ian Head, Newcastle upon Tyne (GB); Eileen Yu, Newcastle upon Tyne (GB); Richard Dinsdale, Newcastle upon Tyne (GB); Alan Guwy, Newcastle upon Tyne (GB); Rodrigo Fernandez Feito, Newcastle upon Tyne (GB); Amandeep Kaur, Newcastle upon Tyne (GB)

(73) Assignee: UNIVERSITY OF NEWCASTLE UPON TYNE, Newcastle upon Tyne, Tyne and Wear (GB)

(21) Appl. No.: 18/293,528

(22) PCT Filed: Jul. 29, 2022
(86) PCT No.: PCT/GB2022/052008

§ 371 Date: Jan. 30, 2024

(30) Foreign Application Priority Data
Jul. 30, 2021 (GB) 2110984.8

(51) Int. Cl. G01N 27/416; G01N 27/327; G01N 33/18; H01M 8/16

(52) U.S. Cl. CPC G01N 27/4166; G01N 27/327; G01N 27/4163; G01N 27/4167; G01N 33/1826; H01M 8/16

(57) ABSTRACT

A water quality monitoring method. The method comprises: receiving BioElectrochemical System, BES, sensor data indicating an output from at least one BES sensor exposed to a water sample; and receiving data indicating at least one environmental parameter, at least one piece of configuration data for the BES sensor or at least one parameter for a system in which the BES sensor is implemented. The received data is processed according to a calibration algorithm to generate a parameter indicative of organic compound concentration for the water sample. A water quality monitoring device to implement the method may comprise a BES sensor and a processor to implement the calibration algorithm, and optionally one or more further sensors.

### TECHNICAL FIELD

The present invention relates to a water quality monitoring method and device. Particularly, but not exclusively, certain examples of the present invention relate to a method and device to determine changes in levels of organic compounds or toxic pollutants in water. Certain examples are applicable to monitoring waste water.

### BACKGROUND

Water is fundamental to human life. Handling our waste water is critical as populations grow at unprecedented rates globally. Without comprehensive water management, we will struggle to protect our environment.

In the UK, river water quality has been declining over recent years. At the same time, fines for pollution incidents have increased. Improved water monitoring can help to detect and stop the discharge of organic pollutants and toxic compounds from waste water treatment facilities.

Current water monitoring systems provide data hours, days or even weeks after testing. For example, Biochemical Oxygen Demand (BOD) is a standard parameter used for water quality. BOD is the amount of dissolved oxygen in water required (demanded) for aerobic biological organisms to break down organic matter present in a water sample (through microbial substrate oxidation). A higher BOD value means that more oxygen is required to breakdown the organic matter in the sample: that is, the sample has a higher level of organic matter. BOD is therefore frequently used as a gauge of the effectiveness of waste water treatment plants, and a BOD value can indicate the short-term impact on the oxygen levels of river water downstream of the treatment plant, and thus the level of discharged organic pollutants.

BOD is usually measured off-line over five days, and a BOD value is normally expressed in milligrams of oxygen consumed per litre of sample during 5 days of incubation at 20° C. This off-line test, or the result of the test, is commonly referred to as BOD₅. Clearly, an off-line test that takes five days to complete means that organic pollutants could be continuously discharged for five days or more before this is detected. A timely response to a pollution event is not possible. This can lead to effluents failing to meet regulatory standards and incidents going undetected, causing health and environmental issues downstream.

Current water monitoring methods suffer from a number of known problems. They may be labour intensive, inaccurate or require frequent recalibration to avoid measurement drift (or any combination). For instance, the BOD₅ test requires a sample to be gathered and returned to a laboratory, extensive preparation/dilution steps and then incubation for five days to measure a dissolved oxygen concentrations. To apply the BOD₅ test across multiple locations presents a logistical challenge. It is also relatively inaccurate, with accuracy typically ±15%.

More recently, BOD sensors have been developed which allow online monitoring with increased speed and accuracy. However, existing respirometric, photometric and mediated electrochemical sensors generally exhibit poor stability, low dynamic range and require frequent maintenance, which has prevented their widespread application.

BOD is not the only measure of the level of organic pollution in water. However, similar tests such as Chemical Oxygen Demand (COD) suffer from similar drawbacks in terms of timeliness and labour intensity. COD is also less specific than BOD for measuring organic matter pollution.

Bacteria found in waste water can break down organic matter. But when toxic compounds are present they can inhibit these bacteria. It is therefore desirable to also measure toxicity (the presence or level of toxic pollutants in water). Toxicity is a difficult parameter to measure, especially in real-time. Frequently, it is not measured at all in waste water treatment plants. Toxicity testing tends to require off-line analysis using fixed microorganism cultures which may not be representative of waste water treatment plant microorganism cultures. Accordingly, test results may not be indicative of how waste water treatment plant microorganism cultures would respond to the measured toxicity.

It is an aim of certain examples of the present invention to solve, mitigate or obviate, at least partly, at least one of the problems and/or disadvantages associated with the prior art. Certain examples aim to provide at least one of the advantages described below.

### BRIEF SUMMARY OF THE INVENTION

According to a first aspect of the present invention there is provided a water quality monitoring method, the method comprising: receiving BioElectrochemical System, BES, sensor data indicating an output from at least one BES sensor exposed to a water sample; receiving data indicating at least one environmental parameter, or at least one piece of configuration data for the BES sensor or at least one parameter for a system in which the BES sensor is implemented; and processing the received data according to a calibration algorithm to generate a parameter indicative of organic compound concentration for the water sample.

The BES sensor may comprise a Microbial Fuel Cell, MFC, comprising an electrode coated with electrogenic bacteria arranged such that as the water sample flows past the electrode, organic matter is consumed by the electrogenic bacteria to generate an electrical current, the BES sensor output being indicative of that electrical current.

The BES sensor data may be indicative of the sum of BES sensor outputs from a plurality of BES sensors arranged hydraulically in series such that the water sample flows through the plural BES sensors or the outputs of each BES sensor individually. For a plurality of BES sensors the BES sensor data may be indicative of normalised current data for each BES sensor.

The at least one environmental parameter data may comprise data indicative of one or more of: the temperature of the water sample; the temperature of the ambient air; the electrical conductivity of the water sample; the pH of the water sample; the dissolved oxygen level of the water sample; or the rainfall recorded in proximity of the sensor.

The at least one piece of configuration data for the BES sensor or measured from the process or system may comprise data indicative of one or more of: water flow rate; external resistance of a BES sensor; electrode size; electrode spacing; chamber volume;

membrane type; catalyst type; biofilm age; maximum power; maximum current; open circuit potential

The at least one parameter for a system in which the BES sensor is implemented may comprise data indicative of one or more of: water flow rate; or water tank level.

The calibration algorithm may comprise an AI algorithm, machine learning algorithm or multiple regression trained according to a data set comprising, for a plurality of water samples, BES sensor data, validation data indicating organic compound concentration for the water samples determined according to an alternative test, and at least one of environmental parameter data or BES sensor configuration data.

The alternative test may comprise at least one of: Biological Oxygen Demand, BOD; Chemical Oxygen Demand, COD; or Total Organic Carbon, TOC.

The method may further comprise: determining a change in BES sensor data between a first time point and a second time point for each of the plurality of BES sensors; and determining, based on the change for each BES sensor, an indication whether the change is indicative of a change in organic compound concentration, the presence of toxicity, excessive organic compound concentration or sensor recovery from excessive organic compound concentration or toxicity.

The method may further comprise: monitoring organic compound concentration based on the parameter indicative of organic compound concentration; or controlling process flows in a waste water treatment plant or industrial process based on the parameter indicative of organic compound concentration in order to maintain organic compound concentration within a predetermined tolerance.

Monitoring organic compound concentration or controlling process flows may be further based on the indication whether the change is indicative of a change in organic compound concentration, the presence of toxicity, excessive organic compound concentration or sensor recovery from excessive organic compound concentration or toxicity.

According to a second aspect of the present invention there is provided a computer-readable storage medium having computer-readable program code stored therein that, in response to execution by a processor, cause the processor to perform the method of any one of the preceding claims.

According to a third aspect of the present invention there is provided an apparatus comprising a processor and a memory storing executable instructions that, in response to execution by the processor, cause the apparatus to perform the method of any one of the preceding claims.

According to a fourth aspect of the present invention there is provided a water quality monitoring device comprising: a BES sensor configured to generate a current output when exposed to a water sample; and a processor configured to receive data from the BES sensor and data indicating at least one environmental parameter, at least one piece of configuration data for the BES sensor or at least one parameter for a system in which the BES sensor is implemented and to execute a calibration algorithm to generate a parameter indicative of organic compound concentration.

The processor may be further configured to perform the above method.

The device may further comprise an actuator coupled to the BES sensor configured to selectively tilt the biosensor upwards or downwards.

The device may further comprise a plurality of BES sensors arranged hydraulically in series such that the water sample flows through the plural BES sensors, each BES sensor being configured to generate a current output when exposed to a water sample.

According to certain examples of the present invention there is provided a new water quality monitoring method and device. Certain examples of the present invention using a BioElectrochemical System (BES) sensor have the potential to reduce the discharge of pollutants from treatment facilities by detecting pollution events in a timely fashion.

Advantageously, the use of a BES sensor allows for real-time water quality monitoring and more accurate process control, for instance for a waste water treatment plant to mitigate against pollution events. Providing accurate, real-time data, permits immediate action and intervention to be taken. Examples of the present invention deliver a data-driven biology solution for waste water treatment plant operators and regulators. In certain examples, the BES sensor achieves this at low cost and with minimal maintenance needs. Furthermore, the detection of organic pollutants or toxic pollutants in water may be less labour intensive than conventional techniques.

Certain examples of the present invention provide a BES sensor capable of monitoring water quality, for organic compound concentration and/or toxicity. Particularly, certain examples of the present invention determine a prediction of organic compound concentration using a BES sensor. Organic compound concentration is a measure of the amount of organic compounds in a water sample, for instance mg/l, whereas organic load typically has units per time (mg/l/day), however these terms may be used interchangeably as if a sensor measures levels of organic compounds then a load may be computed. Where in the present description reference is made to organic load or organic loading, this should be interpreted broadly to mean a measure of organic compound concentration. According to certain examples, the BES sensor data may be incorporated with at least one further piece of parameter data which may comprise one or more of an environmental parameter, at least one piece of configuration data for the BES sensor or at least one parameter for a system in which the BES sensor is implemented. An environmental parameter may include water pH, conductivity, temperature or dissolved oxygen. The following description provides greater detail of these different examples of further parameter data. Combining these parameters in a calibration algorithm with the BES sensor data gives a more precise estimate of the organic loading (water quality/pollution).

According to certain examples, multiple BES sensors may be hydraulically connected in series, thereby enabling toxicity pollution events to be distinguished from organic load pollution events. That is, for a single BES sensor a signal drop arising from BOD-related events (for instance, low BOD/less polluted water) may be indistinguishable from increased toxicity/shock load (more polluted) but according to certain examples of the present invention, multiple sensors permits this distinction to be made.

Another aspect provides a computer program comprising instructions arranged, when executed, to implement a method in accordance with any one of the above-described aspects. A further aspect provides machine-readable storage storing such a program.

### BRIEF DESCRIPTION OF THE DRAWINGS

Examples of the invention are further described hereinafter with reference to the accompanying drawings, in which:

FIG. 1 is a schematic illustration of a Microbial Fuel Cell (MFC);

FIG. 2 is a schematic illustration of BES sensor;

FIG. 3 is an exploded view of the BES sensor of FIG. 2;

FIG. 4 is a graph indicative of the relationship between measured current and BOD₅ for the MFC of FIG. 1 and the BES sensor of FIGS. 2 and 3;

FIG. 5 comprises graphs indicative of predicted BOD₅ for the BES sensor of FIGS. 2 and 3 against separately measured BOD₅ for each of a three-stage series arrangement of BES sensors, and the predicted BOD₅ against measured BOD₅ when the measured currents for each BES sensor are summed;

FIG. 6 comprises graphs showing respectively the response of a three-stage series arrangement of BES sensors to a reduction of organic pollutants and an increase in toxicity;

FIG. 7 is a flow chart showing a water quality monitoring method according to an example of the present invention;

FIG. 8 is a subroutine for the flow chart of FIG. 7 showing the operation of a BES sensor, for the example of an MFC;

FIG. 9 is a subroutine for the flow chart of FIG. 7 showing the operation of a BES sensor, for the example of a Microbial Electrolysis Cell (MEC);

FIG. 10 is a subroutine for the flow chart of FIG. 7 showing the normalisation of current from a BES sensor;

FIG. 11 is a subroutine for the flow chart of FIG. 7 showing the calculation of BOD change or toxicity presence;

FIG. 12 comprises graphs showing the relative accuracy of predicting COD, firstly using BES sensor data only, secondly using water conductivity data only, and thirdly further using inputs of BES sensor and conductivity data combined;

FIG. 13 comprises graphs showing the relative accuracy of predicting COD, firstly using a neural network trained with BES sensor data only, and secondly a neural network trained using inputs of BES sensor data, water flow rate data and other measured environmental parameters (water conductivity, pH and temperature);

FIG. 14 is a schematic illustration of a test rig according to an example of the present invention; and

FIG. 15 is a flow chart showing a water quality monitoring method according to a further example of the present invention.

### DETAILED DESCRIPTION

Electrogenic bacteria can form a biofilm on an electrode surface. As they break down organic matter (which could be found in polluted water), they donate electrons which generates electricity. The present inventors have identified that in a Bio-Electrochemical System (BES) this small amount of generated electricity can equate to the concentration of organic material in water. Conversely, when toxic compounds are present, they inhibit bacteria and the electricity generated reduces.

Because BES sensors operate by biofilm response they do not require clear optical paths. This is an advantage over commercially available fluorescence-based BOD sensors which require an optical view finder and therefore are problematic (subject to fouling and poorly correlate) in turbid/polluted waste waters. Furthermore, non-biological sensors do not provide any toxicity information.

As will be described, BES sensors can also provide a calibrated response within specific thresholds for process control. For instance, by positioning a BES sensor upstream of biological treatment processes allows adjustments to be made to those treatment processes thereby avoiding the need to treat waste water at maximum capacity (assuming the worst-case scenario for organic loading). This can provide for costs savings as well as greater pollution control.

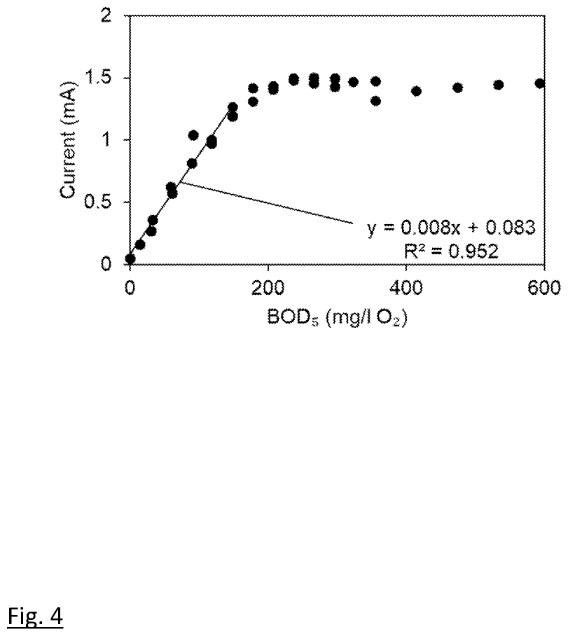

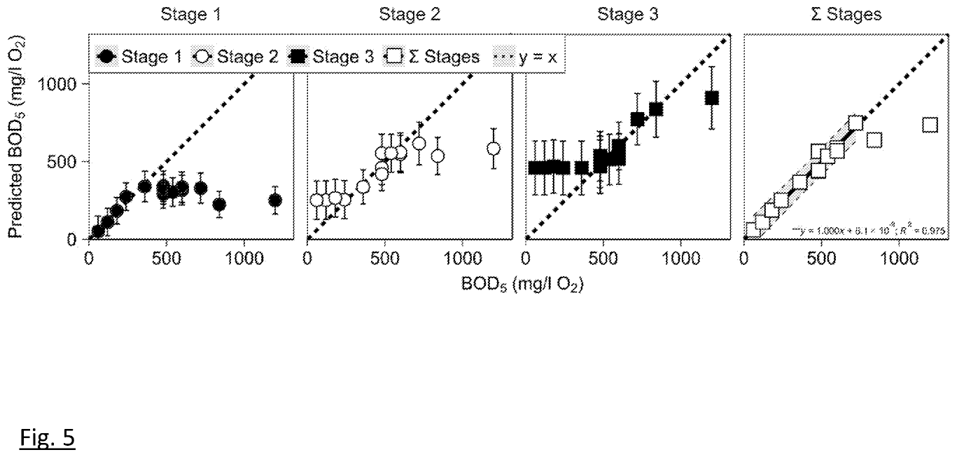

Microbial Fuel Cell (MFC) technology uses microorganisms to convert biochemical energy into electrical energy. The electrical voltage, current or charge generated correlates to the concentration of biodegradable organic carbon (for instance, BOD) consumed at an anoxic anode. An MFC may thus be used as the basis of a BES sensor, though as described later on, BES sensors may take other forms. Where in this document reference is made to BES sensor data this encompasses an output from a BES sensor, for instance an MFC, and may be measured parameter such as voltage, current or charge, a derived value, for instance current if voltage is measured and the resistance is known. BES sensor data may be dimensionless or normalised, for instance expressed as a percentage of a predetermined maximum, as discussed below. Alternative types of BES sensor data will be apparent to the skilled person dependent on the underpinning technology. An MFC is promising for sensing applications because the current generated is stable, responsive and has been found to be proportional to BOD, for at least a limited dynamic range (as described below in connection with FIGS. 4 and 5).

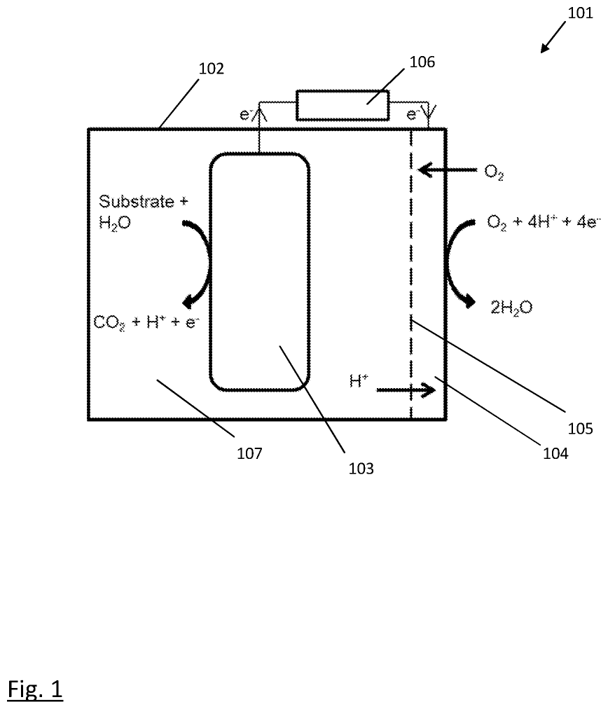

FIG. 1 schematically illustrates an MFC 101 comprising an anaerobic chamber 102 including an anode 103 and an air cathode 104 separated from the anaerobic chamber 102 by an ion exchange membrane 105. In some examples, the membrane 105 may be a polymeric membrane specific for cation exchange, e.g. a gel polystyrene cross linked with divinylbenzene. In other examples, a BES may use anion exchange membranes, ceramics, battery separators, or even have no membrane and just have electrodes separated by distance (which may result in poorer performance). Both the anode 103 and the cathode 104 (which is exposed to air) may be formed from any suitable material which is both conductive (and bio-compatible for biofilm supporting electrodes), such as carbon cloth, and are electrically connected via an external resistance 106. The current flowing through resistance 106 may be measured.

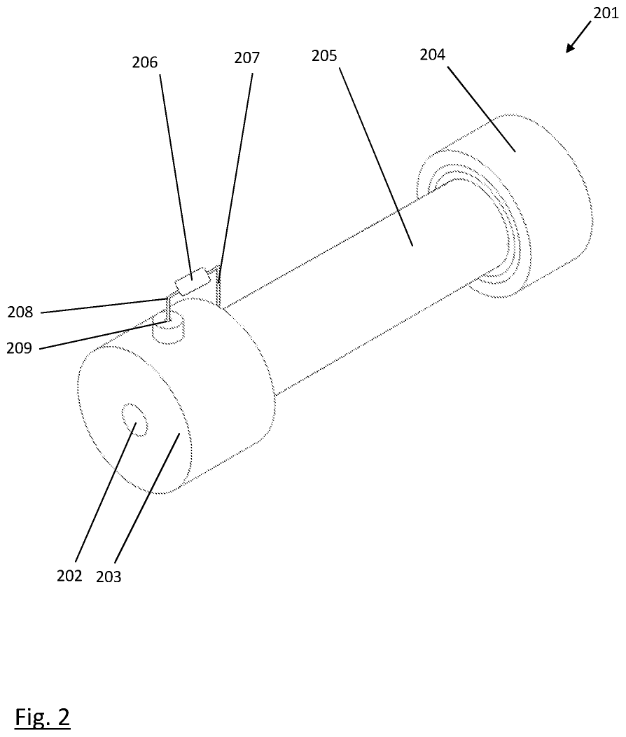

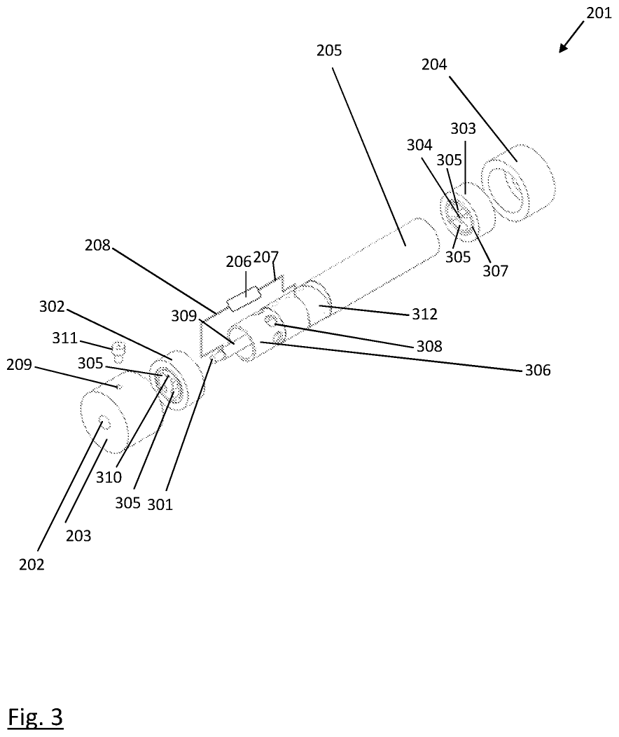

The anode 103 has a biofilm of electrogenic bacteria formed upon its surface. The chamber 102 is filled with a substrate 107 and water. The substrate 107 comprises any suitable organic electron donor which may be oxidised by the bacteria of the biofilm: microbial substrate oxidation converts the substrate 107 and water to carbon dioxide, hydrogen ions (protons) and electrons. For the BES sensors of FIGS. 2 and 3, the substrate comprises organic compounds in the water sample. The dissolved inorganic carbon dioxide is removed from the chamber 102 in the waste stream. The ion exchange membrane 105 permits the hydrogen ions to pass through to the air cathode 103. The external resistance 106 conducts the free electrons as an electrical current. At the air cathode 104 (which may be coated by a chemical catalyst for instance platinum) in a MFC, oxygen reduction takes place, producing water.

Referring now to FIGS. 2 and 3, a BES sensor 201 based upon an MFC is illustrated (in assembled and exploded views respectively). BES sensor 201 is a linear flow sensor: water enters through an inlet 202 in a first end fitting 203 and exits through an outlet (not visible) in a second end fitting 204. The water flowing through may be a sample fraction of the total waste water flow from a waste water treatment plant or between, or upstream of, processes within a waste water treatment plant, industrial process or environmental water body. Water exiting the BES sensor 201 may pass back to the main water flow or may pass to a subsequent similar BES sensor connected in series, as will be elaborated upon in connection with FIGS. 4 to 6.

The BES sensor 201 is generally a tubular construction. Between the end fittings 203, 204 the outermost portion shown in FIG. 2 is a cathode 205 exposed to the air. FIG. 2 further shows an external resistance 206, and current flowing through resistance 206 may be measured and comprises the output of the BES sensor 201. The resistance 206 is coupled via a first wire 207 to the cathode 205 and via a second wire 208 to an anode (not visible in FIG. 2) via a connection hole 209 in the first end fitting 203.

Turning to FIG. 3, the internal parts of the BES sensor 201, and its operation, will now be described. The working components of the BES sensor 201 are supported by a rod 301 that extends between bungs 302, 303 (including inward facing holes 304 to receive the rod 301) that in turn are received by the end fittings 203, 204. Bungs 302, 303 include slots 305 so that water may pass through. Surrounding the rod is a tubular spacer 306 that is received in an annular groove 307 in bungs 302, 303. The spacer 306 includes orifices 308 so that water flowing through the sensor 201 within the spacer 306 is in fluid connection with components both inside and outside the spacer 306. Bungs 302, 303 may be threaded to be secured in corresponding threaded bores in end fittings 203, 204. Similarly, the rod 301 may have threaded ends to secure within holes 304, which on assembly pulls the end caps 203, 204 together and sandwiches tube 306 into grooves 307 (containing O-rings, not shown to seal the cell). It will be appreciated that other construction methods may be used, including a single piece construction for end fittings and bungs, or instead of a rod the outer wall of the tube could be an anode-membrane-cathode. The present invention is not limited to any specific construction of the BES sensor 201.

An anode 309 is formed about rod 301. Anode 309 may comprise a carbon cloth. A biofilm (not illustrated) is formed upon anode 309, and anode 309 is connected to second wire 208 through a hole 310 in bung 302. A bung 311 seals connection hole 209 in end fitting 203 about second wire 208. An ion exchange membrane 312, which may be formed from the same materials noted above for FIG. 1, is wrapped about the outside of spacer 306. The cathode 205 is wrapped about the outside of the membrane 312. Cathode 205 may comprise a platinised, wetproofed carbon cloth.

Anode 309 and cathode 205 are thus separated by membrane 312. Water can flow continuously through the BES sensor 201, particularly through tubular spacer 306 which defines an anaerobic chamber housing the anode. The electrical current flowing through resistor 206 comprises a real-time output indicative of water quality, and particularly indicative of biodegradable organic matter (often measured as BOD), as described below in connection with FIG. 4. The electroactive biofilm (bioreceptor) grown upon anode 309 oxidises organic compounds found in organic pollution which may be present in a waste water sample. An anode with a biofilm in a BES sensor 201 has been demonstrated to operate continuously for over three years without replacement, and to have a stable calibration over this time.

The air-facing cathode 205 reduces oxygen to water. As the anode 309 and cathode 205 are connected electrically, a current is generated as organic compounds are oxidised at the anode and oxygen reduced at the cathode. Therefore, the BES sensor output signal is responsive to changes in concentration of biodegradable organic compounds (equivalent to measured BOD): when the concentration of biodegradable organic compounds is higher, more electricity is generated. Conversely, when toxic compounds are present in the water stream they inhibit the anodic biofilm and the output electrical signal decreases.

The current output from a BES sensor 201 may be calibrated to independently measured BOD₅ values for water samples with different organic substrate concentrations. FIG. 4 shows measured current data for a BES sensor 201 fed with a sample of synthetic waste water containing glucose and glutamic acid as an organic substrate. The BES sensor was operated in batch mode; that is, with the sample static within the sensor. BES sensor calibration was performed by measuring the peak output current for each substrate concentration (and for each sensor) and then replacing the sample with one having a different substrate concentration and repeating the peak current measurement. The sample was also tested off-line according to the standard BOD₅ test.

FIG. 4 shows a linear relationship between peak current and BOD₅ concentration until approximately 178 mg/l. At this point this particular anode biofilm becomes saturated with substrate and the current response plateaus. Below this concentration the sensor can be used to determine unknown BOD values with a standard deviation of ±8%. This compares favourably with the accuracy of the standard BOD₅ test which has a standard deviation of ±15%.

A linear response up to a BOD concentration of 178 mg/l is valuable: a current output that has plateaued provides useful information that a waste water sample includes organic pollutants of at least 178 mg/l. However, it does not provide a sufficient dynamic range to accommodate real life waste water samples, and a larger dynamic range is desirable for high strength industrial effluents.

The present inventors have established that by using a multi-stage BES device, it is possible to extend the dynamic range of the sensor. This expands the applicability of BES sensing to high strength waste waters (that is, waste waters with a significant organic load). Turning to FIG. 5, this illustrates as an example the current outputs from each BES sensor in a three-stage, single-pass, flow-mode BES device when fed with the same synthetic waste water as before. This BES device comprises three BES sensors 201 according to FIGS. 2 and 3 connected hydraulically in series, such that the first stage is the first to receive waste water and the third stage is the last to receive the waste water. Hydraulically connected MFC based BES sensors possess unique characteristics because unconsumed substrate from the first stage flows to subsequent stages where the anodic biofilms are acclimated to lower substrate concentrations. For each stage, FIG. 5 shows a graph of predicted BOD₅ (from the BES sensor output current) against estimated BOD₅ for synthetic waste water samples at different concentrations. Estimated BOD₅ is measured off-line for each synthetic waste water sample concentration according to the standard BOD₅ test.

For the three BES sensors connected hydraulically in series in this example the concentration of organic matter is reduced by the action of each sensor in turn. Consequently, for an initial BOD level that is outside of the dynamic range for a single BES sensor, for the second and third sensors the BOD level has been reduced to a level for which the result is linear. In FIG. 5 the graph for each stage shows a different BOD range for which an approximately linear response is given between predicted and estimated BOD₅. For the first stage, generally the same linear relationship followed by plateau from FIG. 4 was exhibited in the current response as the organic matter concentration increased. However with the second and third stages the current did not begin to rise until after the first cell had reached plateau current (essentially the first stage having consumed the majority of the organic matter up to the plateauing point) and so the linear response portion corresponds to a different part of the absolute BOD₅ range.

The present inventors have identified that by first summing the output current from each BES sensor in the series a linear response over a significantly wider dynamic range may be established for the three-stage BES device as a whole. In particular, the normalised current densities from each stage are summed which eliminates the effects of non-sensing BES parameters (such as cathode performance as it degrades over time, membrane fouling and architectural, structural or electrical differences between cells), thus allowing the contribution of each stage to be validly combined. By taking the sum of the current generated and plotting against estimated BOD₅ it is evident that a linear correlation continues, and the dynamic range of this sensor is extended significantly (up to 723 mg/l). Average response time to reach a stable current for reliable quantification of the output current was 2.3 hours, which represents a significant improvement on the standard BOD₅ test and may be referred to as near real-time. Comparing the multi-stage, flow-mode sensor with other MFC based BOD sensors reported in the literature it is clear that the range is considerably improved. Advantageously, this allows the BES device to be used for high strength industrial effluents as well as lower concentration municipal waste water.

It will be appreciated that a three-stage BES device is merely exemplary. An improved dynamic range may be provided with two stages (for instance, for lower strength/less polluted waters), and indeed four or more stages may provide further improvements (such as with high strength industrial waste waters). Three-stages have been found to give a practical balance between a useful dynamic range and cost or complexity of the system in monitoring of municipal settled sewage.

As previously noted, for a single stage BES sensor a decrease in electrical current may be due to either a decrease in organic concentration (a reduction in BOD) or the introduction (or increase in concentration) of a toxic compound-both induce the same effect on the sensor output current. Without independent verification of toxicity, a reduced electrical current might be interpreted as a reduction in overall waste water pollution, whereas in fact the reverse might be true. The present inventors have identified how a multi-stage BES device can be used to establish whether a reduction in the summed electrical current is due to a decrease in organic pollution, requiring less treatment, or an increase in toxicity, requiring more. While a change to both variables simultaneously could remain difficult to interpret, this is unlikely to occur in practice and as explained below the order and timing of response for each stage may still be indicative (for instance if the first stage responds first this may be noted to indicate toxicity. Additionally, it may be expected that a toxic event would last longer than a BOD change and so the length of a BES sensor response may be indicative. Operator evaluation may further determine the likely nature of an unusual response, and indeed a feature of examples of the present invention is that real-time monitoring may be used to trigger an alarm to enable operators to manually investigate and if necessary perform independent testing. A further option in some examples of the present invention is to operate the system such that at least one initial BES stage is always operated in a saturated state (as explained below) for instance by manipulating flow rate or external resistance so that for even the lowest expected BOD levels that cell essentially becomes a toxicity only sensor.

To achieve this differentiation between BOD change and toxicity change, the same BES device as described above is used, including multiple BES sensor cells connected hydraulically in series. In addition to considering the current output of each stage (or the peak output current in a measurement time window) changes in current output for each stage are noted and the order in which the current output from each stage changes is considered. By taking account of the order and magnitude at which a BES stage responds, it is possible to distinguish water quality events.

This order of response information may be considered qualitatively for the example of a two stage BES device, with a first BES sensor and a second BES sensor connected hydraulically in series. It will be appreciated how this may be extensible to devices with a larger number of stages.

When BOD (organic compounds concentration) increases in the incoming waste water, the current output signal increases first for the first stage BES sensor. Some consumption of organic compounds will occur within the first stage sensor). If organic compounds remain in the flow of waste water from the first stage, an increase in current output signal will subsequently be observed in the second stage BES sensor. This is because only when the ability of the first stage to consume the increased organic compounds is exceeded will an excess be passed to the second stage.

Conversely, when BOD decreases, the second stage BES sensor will “starve” first and exhibit a drop in output current (and the signal drop of the second stage may be greatest) as BOD will first be consumed in the first stage BES. That is, for any change in BOD, the first and second stages will respond at different times, and the magnitude of any signal change may differ.

When toxicity increases, the first stage BES will be inhibited to some extent (and thus consume less BOD), and the second stage BES will then be inhibited to the same extent (as toxicity is not consumed by the first stage), manifesting in an output current decrease at substantially the same time.

Conversely, for a reduction in toxicity the degree of inhibition of the bacteria in each stage will reduce, manifesting in an output current increase at substantially the same time. Accordingly, the time of any signal change will be substantially the same for each stage in the event of a change in toxicity, with any time offset resulting only from flow rates through the device.

Where there is a very high increase in BOD (resulting in very high absolute BOD levels), the first stage BES output current will be inhibited most (greatest signal drop) as non-electrogenic processes dominate. For the example previously given for testing with glucose and glutamic acid synthetic medium, at very high levels there is an inhibition of the signal generation in the first MFCs. In this case (with this medium) there may also be observed significant production of biomass sludge. DNA/community analysis has determined that at these concentrations the abundance of bacteria associated with glucose-fermentation (for instance, Lactococcus) significantly increased (up to 90% of the microbial community). In this case the electrogenic bacteria were overwhelmed by non-electrogenic bacteria (for instance, fermenters, heterotrophs using oxygen permeated through membrane, etc). These non-electrogenic bacteria still consume organic compounds but no electrical current is generated by their processes. Thus the second and third BES stage receive water with a lower BOD which does not overwhelm them (as much). This scenario is essentially a sub-category of the toxicity sensor and would be exactly the same response for a “biodegradable toxicant”: for instance, if the signal-inhibiting compound(s) are degraded through the cascade of BES reactors. As the organic matter will be partially consumed by the first stage, the effect (signal drop) in the second stage BES will be lessened.

In general, if the largest signal drop is observed in the first BES sensor, this is indicative of increased pollution (BOD excess or increased toxicity). Conversely, if the largest signal drop is observed in the second BES second, this is indicative of reduced pollution (a BOD decrease).

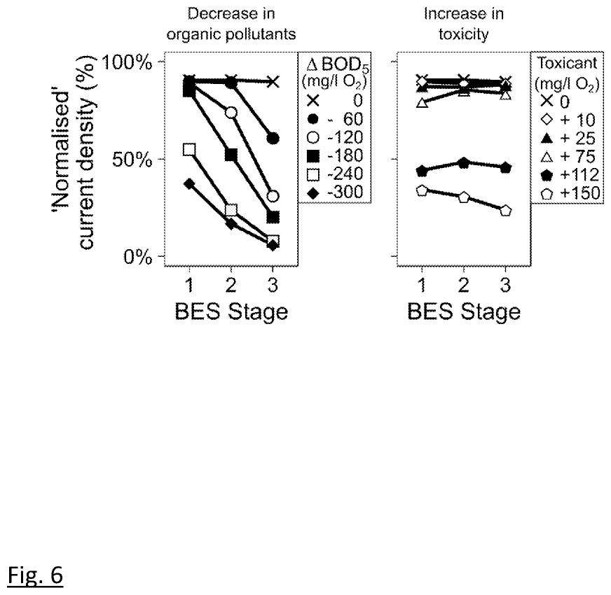

Referring now to FIG. 6, this differentiation of BOD changes and toxicity changes is graphically represented for a three-stage BES device. The left graph shows the response to a reduction in organic pollutants in incoming water. The right graph shows increasing toxicity. On each graph the X axis identifies each stage of the three-stage device (stage 1 being the first stage to receive the water) and the Y axis shows the degree of inhibition (percentage reduction in output current).

For the left hand graph showing the change in inhibition as BOD₅ levels are progressively reduced from 360 to 60 mg/l O₂, and the degree of inhibition is relative to the current output for that stage for 360 mg/l O₂. For each successive reduced BOD₅ concentration the current density for each stage was measured and the minima current density found during a 1.5-hour window within which that BOD₅ concentration was supplied to the first stage. It can be readily seen that the degree of inhibition is greatest for the third stage for each BOD₅ concentration, and it not until a lower BOD₅ concentration water sample is supplied that the first and second stages reach similar levels of inhibition: the last stage responds to a reduction in organic matter first and with the largest degree of inhibition.

For the right-hand graph 4-nitrophenol (as a model toxicant which could be easily measured spectrophotometrically in the laboratory) was introduced in progressively stronger concentrations as a model toxic compound within a synthetic waste water sample with constant concentration of organic matter. The concentration of 4-nitrophenol was progressively increased from 0 to 150 mg/l 4-NP, and the degree of inhibition is relative to the current output for that stage for 0 mg/l 4-NP. For each successive increased toxic concentration the current density for each stage was measured and the minima current density found during a 1.5-hour window within which that toxic concentration was supplied to the first stage. It can be readily seen that the degree of inhibition is more uniform across the three-stages (though for some toxic concentration levels the later stages are more or less inhibited than the first stage).

Accordingly, the magnitude and ordering of the response allowed a decrease in output current signal from decreasing BOD to be distinguished from the presence of a toxic compound. Furthermore, the BES sensors were found to be capable of recovering to previous performance levels within hours of acute exposure to toxicity. The multi-stage BES device configuration therefore enables truly combined BOD and toxicity sensing with enhanced detection capabilities without the need for a defined or synthetic reference feed.

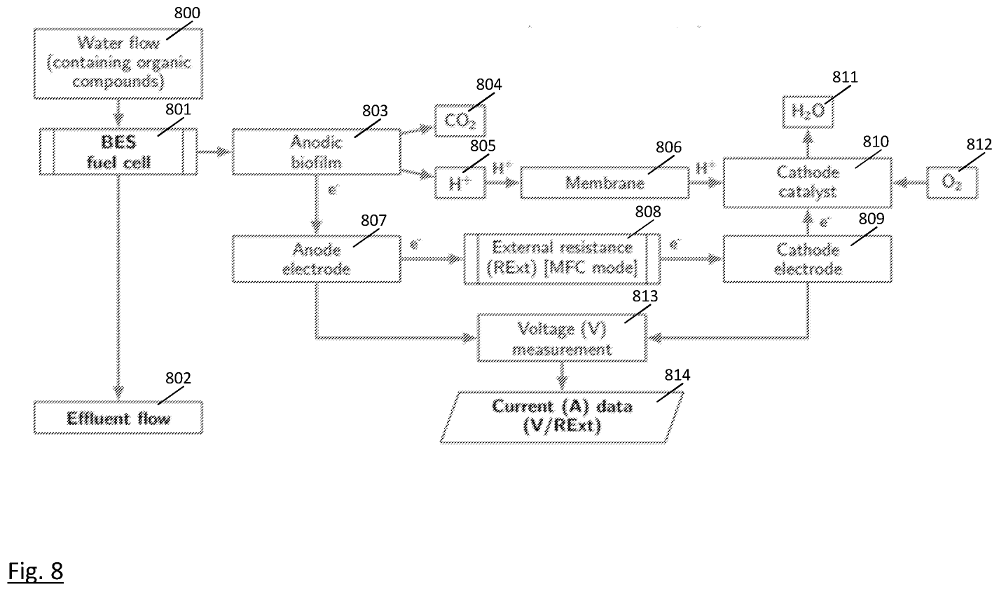

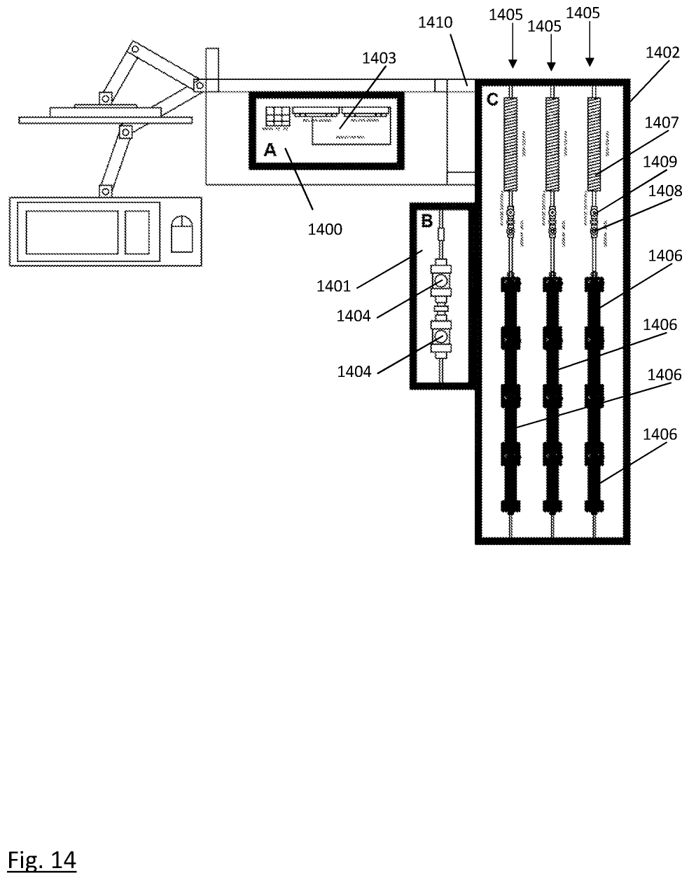

As noted above, a BES device comprising a plurality of series connected BES sensors may be used to monitoring high strength waste waters (until substrate levels becomes inhibiting), without requiring amendment, dilution or aeration. It may further differentiate between BOD changes and toxicity changes. However, when fed real environmental, process or waste waters the sensor performance will be affected by a range of environmental parameters, which may include one or more of water conductivity, water temperature, water pH, dissolved oxygen levels and the composition of the BOD. These variables may jointly or severally change the biosensor activity (all have effects on biological processes). In order for a BES device according to an example of the present invention to be provide accurate information, and particularly in order to be used effectively for process control (such as in a waste water treatment plant) it is necessary to take account of environmental factors when deploying BES sensors in the field. This may be through efforts to control environmental factors, for instance by controlling the temperature of waste water fed to the BES device, or by controlling flow rates through the device through a suitable pump, as described below in connection with FIG. 14. As well or instead, according to certain examples of the present invention, in addition to BES sensor measurement, a water quality monitoring apparatus includes at least one further sensor (or includes an input to receive a measurement from a separate sensor) of at least one environmental parameter from a group including water pH, water conductivity, dissolved oxygen and temperature. In some examples of the present invention, BES sensor parameters may also be used as part of the calibration algorithm to be described below, for instance flow rate, external resistance (see for instance parts 808 and 809 of FIG. 8) or temperature set point. This may be referred to in the following description as BES configuration data.

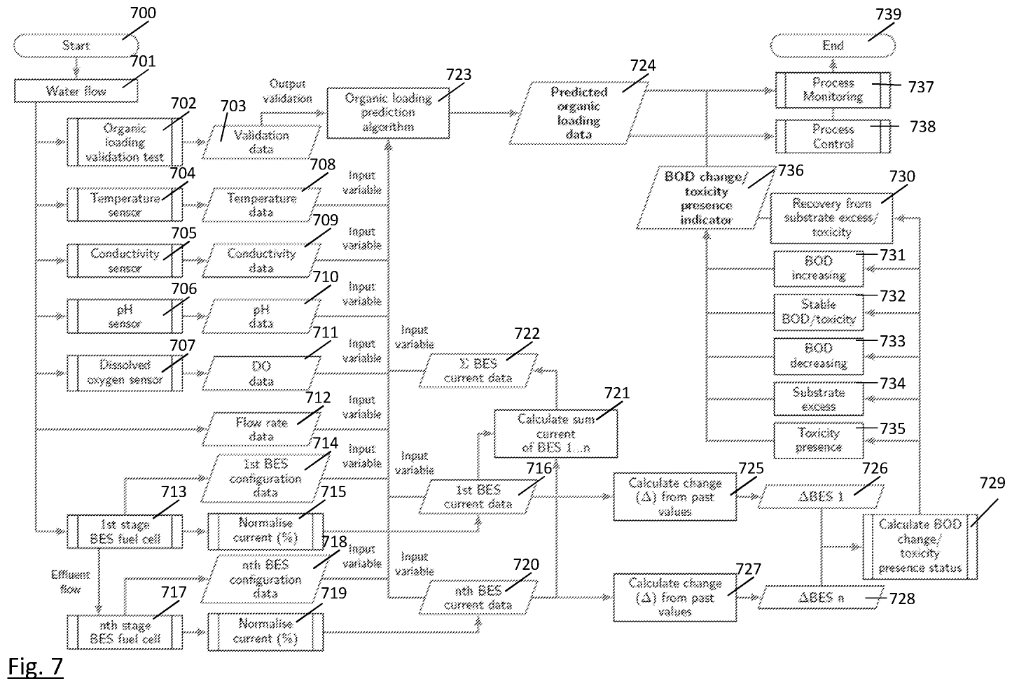

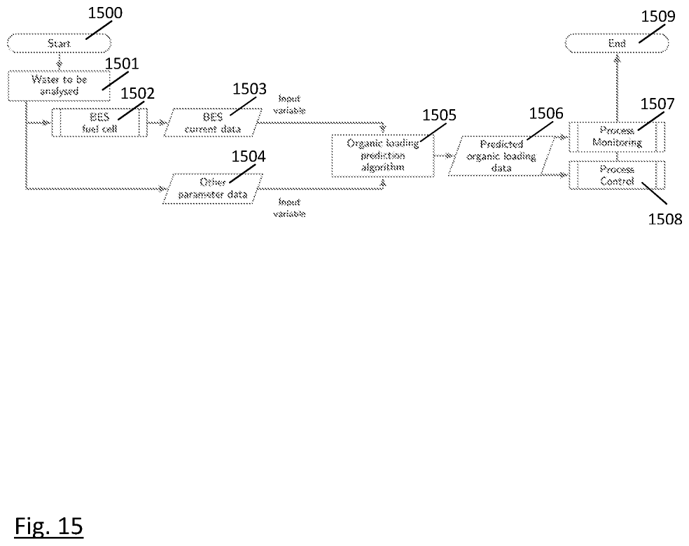

Referring now to FIG. 7, this is a flow chart illustrating a water quality monitoring method according to an example of the present invention. FIG. 15 described later in the present document provides an alternative, simplified illustration of a water quality monitoring method. The method combines data indicating the output from at least one BES sensor (or as illustrated, a plurality of BES sensors) with at least one piece of parameter data which may comprise one or more of an environmental parameter, at least one piece of configuration data for the BES sensor or at least one parameter for a system in which the BES sensor is implemented. An environmental parameter may be measured by at least one further sensor. The method establishes as an output an estimation of a near real-time water quality parameter, which in certain examples may be correlated to desired organic compound concentration measurement, for instance against calibrated off-line measurements of BOD). The flow chart of FIG. 7 further illustrates how according to certain examples of the present invention the BES sensor outputs may be used to establish whether a BOD or a toxicity related event has occurred.

The flow chart of FIG. 7 starts at step 700 and at step 701 water is supplied though the BES device. The BES device comprises at least one BES sensor according to FIGS. 2 and 3. Optionally, there may be at least two BES sensors, and this is required where BOD changes and toxicity changes are to be differentiated. The water supplied to the BES device at step 701, and for which analysis is undertaken, could for instance be municipal or industrial waste water influent or effluent to or from a treatment plant, a process flow within a treatment plant, or any other aquatic source (for instance, river, lake water, seawater or industrial reactors).

The water supplied at step 701 may be a sample from a static source, analysed by the BES device operating in batch-mode. However, it will be appreciated that this would prohibit time-based changes from being measured and also would not allow the enhanced detection range or discrimination of inhibition/toxicity achieved by water flowing through multiple BES sensor stages. The water supplied to the device will have an associated flow-rate 712 (for instance in ml/min), which as explained below may be used as an input for the organic compound concentration prediction algorithm at step 723.

As noted above, a BES device and an organic compound concentration prediction algorithm according to examples of the present invention may be used without calibration against a specific test off-line test. In an uncalibrated mode operating thresholds and parameter ranges are defined for process control/monitoring, optimisation and alerts based on raw outputs from the BES sensors and the environmental sensors. It will be appreciated that if it is known that BES sensor outputs may be equated to organic load data, then it is not essential that they be calibrated against some form of off-line test: the absolute values and time-based changes suffice to the absolute value will suffice for process control and alerts in the event of a pollution event. However, optionally, at step 702 an independent organic compound concentration validation test may be performed on water supplied at step 701.

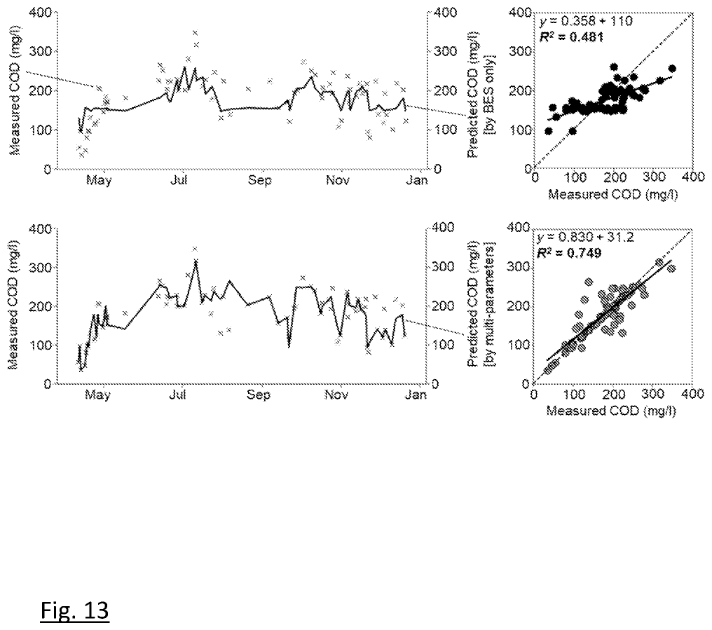

Typically, step 702 will comprise taking a sample of the water supplied at a known time and running a test off-line, for instance for BOD₅, Chemical Oxygen Demand (COD), Total Organic Carbon (TOC) or any other standard test indicative of organic load in water, as will be known to the skilled person. This independent measure of organic loading may be compared against the output of the organic loading prediction algorithm (described below) and used to provide calibration in terms of a standard measure of water quality. The output of step 702 is organic loading data at step 703. For instance, this may be a BOD₅ value in units of mg/l O₂. The comparison and hence calibration of independently measured BOD₅ and predicted organic loading is shown in FIG. 13.

As noted previously, a BES sensor output may be correlated to a BOD parameter or may be used as-is with the electrical current as a proxy parameter for water quality (if step 702 is omitted). However, a BES sensor output may also change in response to in other measurable parameters such as temperature, conductivity, pH and dissolved oxygen, each of which can affect the biology and therefore the biosensor signal. According to certain examples of the present invention, the water quality monitoring apparatus may include one or more of a water temperature sensor 704, conductivity sensor 705, pH sensor 706 and dissolved oxygen sensor 707 (or alternatively include one or more sensor inputs to receive values from separate sensors). Further parameters relating to a system in which the BES sensor is implemented, for instance a waste water processing plant, may also be used as well as or instead of environmental data. For instance, the flow-rate of the water 712 to be analysed a water tank level within part of the system may be a suitable input. More generally, and data associated with a water processing system or in a treatment plant may suitably be used as an input. Again, as well as or instead of an environmental parameter or a system parameter, in certain examples of the invention BES configuration variables 714, 718 (such as the external resistance in an MFC or applied potential in a MEC, electrode size, electrode spacing, chamber volume, membrane type, catalyst type, biofilm age, maximum power, maximum current or open circuit potential) may be used in combination with BES sensor data within the prediction algorithm 723. The skilled person will appreciate how such process variables can affect the biosensor signal. Indeed a wider range of sensor inputs might also be provided as an input to the algorithm at step 723, for instance data indicating local rainfall levels or ambient air temperature. As an example, the present inventors have identified how local rainfall data may be correlated with BES sensor data and used to improve its accuracy in predicting organic compound concentration. Other factors that might provide suitable inputs to the algorithm will be apparent to the skilled person from the disclosure herein, knowledge of the specific sensing apparatus deployed to implement the present invention and the particular industrial, commercial or environmental application to which the example implementation of the invention is addressed. The present inventors have determined how the inclusion of one or more of these parameters as inputs to an organic loading prediction algorithm can provide more accurate predictions. Particularly, this enables a significantly improved calibrated signal which correlates more closely with the desired validation parameter in more diverse environments under a broader range and variable environmental conditions.

It will be appreciated that not all environmental sensors 704-707 are needed in combination—each is optional. However, in some examples, each further inclusion of an environmental sensor provides a further increase in accuracy of prediction. In some examples, BES sensor data may be used on its own within the organic loading prediction algorithm. Sensors 704-707 provide (either within device or separately as part of another process monitoring system feeding in the data) respectively temperature data 708 (for instance, in ° C.), conductivity data (for instance, in μS/cm), pH data (for instance, in pH units) and dissolved oxygen data (for instance, in mg/L).

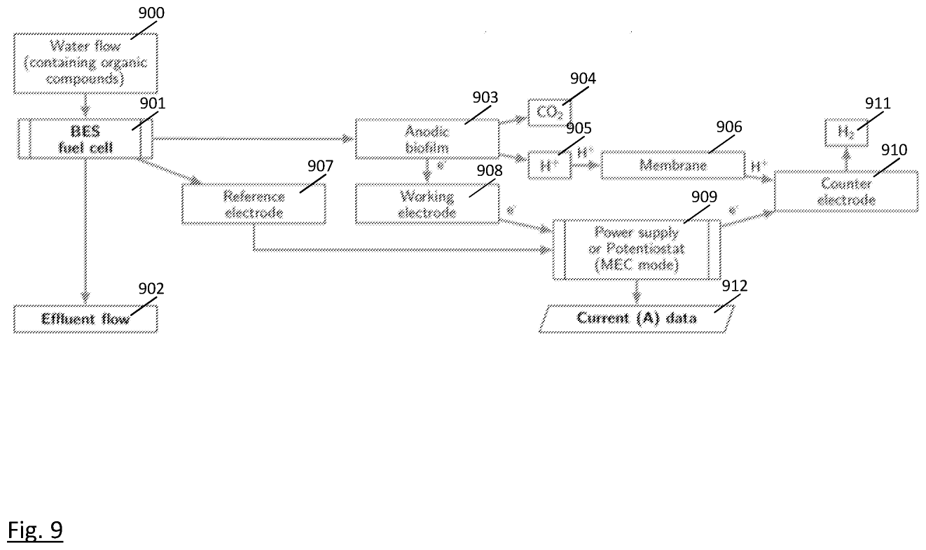

At step 713 the water supplied at step 701 passes to a first stage BES sensor (the first BES sensor receiving the water flow). The water may already be flowing or may be actively pumped, for instance using a peristaltic pump. The BES sensor at step 713 may be based on an MFC, as described above and illustrated in FIGS. 1 to 3. This may be referred to as a BES fuel cell and is further described below and illustrated in the sub-routine of FIG. 8. FIG. 9 illustrates a sub-routine for an alternative BES fuel cell, operating in MEC mode. The BES fuel cell will have associated BES configuration variables 714, 718 (such as the external resistance in an MFC or applied potential in a MEC), which could be inputted to improve calibration across different operating conditions. The output of the BES fuel cell is first stage BES current data, for instances in Amps. Effluent from the first stage BES fuel cell flows to the next stage BES fuel cell (the n^{th} stage BES fuel cell 717 being illustrated).

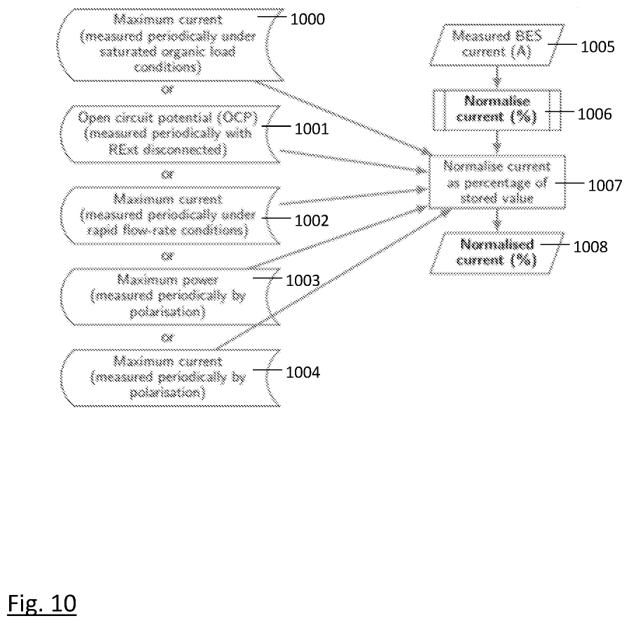

The output of the BES fuel cell 713 is a current signal, and at step 715 the BES current is normalised to take account of performance differences between each stage BES. As an example, this normalisation may comprise expressing the current output as a percentage of a stored parameter, for instance the max current output for the BES fuel cell. This normalisation is further described below in connection with the sub-routine of FIG. 10. The output of the normalisation process is first stage BES current data 716, which may be expressed as a percentage.

While the prediction algorithm could still give accurate prediction with a single BES sensor, optionally combined with a single environmental parameter, in certain examples a plurality of BES sensors is illustrated. Furthermore, multiple stages are required for increased dynamic range and to discriminate BOD change from the presence of toxicity. Only the last, n^{th} stage 717 is illustrated, which receives the effluent from the n^{th-1} BES. The number of stages may depend on the process being monitored: more BES stages leads to a greater dynamic range measurement by the prediction algorithm before saturation/max current is reached. However, too many BES stages in a low-concentration feed could lead to starvation of the later-stage BES (as organic load will be completely biodegraded). The n^{th} stage BES sensor 717 may be generally the same as for the first stage 713 described above, except that its effluent may be discharged from the BES device, for instance to a process within a waste water treatment plant, or into the environment. The output of the n^{th} stage BES sensor 717 undergoes the same process of normalisation 719 to generate output data 720 as before.

At step 721 the sum of the normalised current output from each BES sensor stage (713 to 717) is calculated from the sum of normalised currents 716, 720. Where each normalised current 716, 720 is expressed as a percentage, the sum 721 remains expressed as a percentage. Optionally, it may be rescaled, for instance divided by the number of BES sensor stages, though this is not required as this parameter is an intermediate value which is an input to the prediction algorithm 723 and is transformed through that algorithm, as described below, to the units of the validation test. The output of step 721 is the sum of the normalised current data, 722 expressed as a percentage of a stored parameter (for instance, the maximum summed current).

Step 723 comprises the organic compound concentration prediction algorithm. The algorithm requires as an input the output of a single BES sensor 713 (when only one is used), normalised data from multiple sensors (716-720), and/or the sum of BES current data 722 (optionally expressed as a percentage) for a multi-stage BES device. That is, the (optionally, normalised) output of one or more BES sensor stage may be used as an input as well as or instead of the sum of BES current data, as illustrated in FIG. 7. The computation of a sum of BES current data is not an essential feature of the present invention. Organic loading validation data 703 for output calibration and validation, and environmental parameter data 708 to 711, reactor configuration data including water flow-rate 712 and a BES configuration value (or values if different) 714, 718 are optional inputs to step 723.

The skilled person will appreciate that there are multiple possible variations on the data input to step 723, the minimum requirement being at least one parameter indicative of the current output from a BES sensor and at least one further parameter that may be used to improve the prediction. The at least one further parameter may be indicative of an environmental parameter (one of 708-711). Alternatively or additionally it may be one or more piece of reactor configuration data, 714, 718 or system data 712. Beyond this minimum requirement, multiple options exist. For instance, if a sum of current data is used for multiple BES sensor stages, this may mean that all BES sensors are considered to use the same configuration and so if configuration data is to be included then only an single input of configuration data would be made, rather than multiple instance of the same configuration data.

The algorithm of step 723 returns as an output predicted organic compound concentration (also referred to as organic loading data) at step 724. Where validation data is not included as an input then the output predicted organic concentration data may be dimensionless. As previously discussed it will still provide an indication of relative level of organic concentration, and an indication of time-based changes in organic concentration (noting that as described below, a determination is first made of whether the change in the output from algorithm 723 is indicative of a change in organic concentration or a change in toxicity). This indication of relative loading and changes in relative loading may be used to perform process monitoring and/or process control as described below in connection with steps 737 and 738.

However, where validation data is included as an input, then the output of the algorithm at step 724 may be expressed in the units of the corresponding validation test. For example, where the validation data is for BOD₅ then the predicted organic loading data at step 724 will be in mg/l O₂.

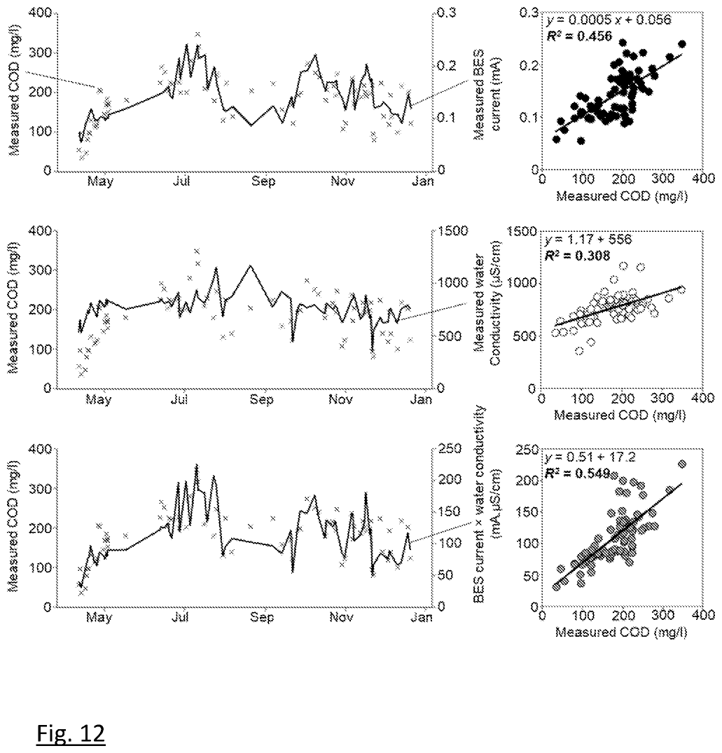

The organic compound concentration prediction algorithm of step 723 transforms the inputted data and outputs a parameter associated with organic loading (either a prediction or uncalibrated signal) and may be based on an Artificial Intelligence (AI), machine learning algorithm or multiple regression techniques. This may be trained on the basis of validation data to determine a predicted organic loading using the BES data and any of the environmental and/or reactor parameters for known off-line test validation data. Alternatively, a more simplistic equation may be selected with a combination of BES data (either sum or individual measurements) with other parameters to derive an organic loading parameter with meaningless units which is not calibrated against an off-line validation test. An example of this is presented below in connection with FIG. 12. Such an equation may be any mathematical operation, for instance multiplication, and may be derived in a laboratory or factory or on another device, then applied directly in the field.

Referring now to FIG. 12, three graphs show the correlations of off-line COD validation data 703 against the BES current data (first line, for at least one BES cell 713), water conductivity data (second line, from conductivity sensor 705) and a combination parameter (BES current multiplied by water conductivity). This is presented as an example for which the prediction algorithm 723 is purely mathematical and not reliant on AI, for instance. FIG. 12 shows an uncalibrated/unvalidated example as no COD data is required to generate the model or the output signal: a simple equation is used. The left hand graphs show over a period of months frequently sampled COD validation data (shown as crosses, relating to the left hand y-axis) and the correlated parameter (shown as a line, relating to the right hand y-axis). The right hand graphs show the linear correlation between COD validation data and each parameter. The upper graphs show data for the summed BES current (of a 8-stage BES cascade) 722 which is correlated with R² of 0.456. The middle graphs show data for the water conductivity 709 which correlated with R² of 0.308. It can be seen that the line correlates best with COD (R²=0.549) for the combination parameter in the bottom graphs for the sum BES current multiplied by the conductivity (as an example of an uncalibrated/untrained combination model). This correlation is presented here to provide clarity and show improvement in correlation, but in this use case the validation (COD) data 703 is optional (unrequired) and the device could be used with only BES and conductivity data to generate the output signal. This output signal could then be fed into process monitoring 737 and control 738 systems for application (for example, alarm threshold/trend analysis).

Turning to FIG. 13, this is a pair of graphs showing the output predicted organic loading data from a prediction algorithm 723 based on the use of a neural networks of step 724 relative to off-line COD validation data 703. The left hand graphs show over a period of months frequent COD validation data (shown as crosses) and the prediction COD output (shown as a line). The right hand graphs show the linear correlation (shown as a solid line) between COD validation data and predicted COD (shown as points) with an ideal prediction (y=x; slope of 1.0) shown as a dashed line. The upper graphs show data for a predicted organic loading from a neural network based on an input of only summed BES current data (from a 8-stage BES cascade). It can be seen that the line is poorly correlated with the validation data (R² of 0.481 and slope of 0.358). The lower graph shows for the same validation data an improved prediction of organic loading from a neural network where BES sensor data is taken into account alongside further parameters including the water flow rate and environmental parameter data (water conductivity, temperature and pH). It can be seen that the correlation is much closer with an R² of 0.749 and slope of 0.830 (closer to ideal of 1).

The neural networks used to generate the results of FIG. 13 were set up with a training dataset consisting of validation data (soluble COD values in mg/l measured from samples taken periodically from the primary settled waste water being fed to the sensing system). The dataset further contained values from sensors measuring data of the same wastewater including water temperature, water conductivity and water pH, and the BES sensor data was the sum current calculated from measurement of an 8-stage BES cascade which were all operated with a fixed external resistance of 268Ω over the training period (thus the external resistance or BES configuration data 714, 718 was not included in the training).

Data in the example dataset (77 values) were normalised between 0-1 by dividing by the maximum value of each parameter in the dataset to be tested. A random sample of 80% of the values (62 values) were used as the training dataset with the remaining 20% used for validation.

For each neural network a feature-wise normalisation layer of the training dataset was then constructed by coercing input(s) into a distribution centred around 0 with standard deviation of 1 (by pre-calculating the mean and variance and calculating (input-mean)/sqrt(var)).

A sequential model was then built comprising this normalisation layer (of input(s)), two hidden, nonlinear, densely-connected layers of 64 output layers using the ReLU (rectified linear unit) nonlinearity activation function, and a linear single-output layer of predicted values. Each model was fitted with a validation split of 0.2 over 3000 iterations (epochs).

The skilled person will readily appreciated that the above described neural network approach is merely an example and in other examples of the present invention any suitable AI, neural network, machine learning or multiple regression technique may be suitably used.

Turning back to FIG. 7, the process of differentiating between a change in organic loading and a change in toxicity will now be described. At step 725 the normalised current data from the first stage BES sensor is processed to determine a change from past values. As an example, the change in the normalised current may be calculated in relation to at least one stored past value from a prior set period (for instance, 1 minute earlier, 15 minutes earlier, or 1 hour earlier). This may be done with a single point value or a rolling average taken over a time period contained within the change interval mentioned above and calculated continuously in real-time as new data arrives. This change is positive if the normalised current has increased since the earlier time point and negative if the normalised current has decreased. The normalised current data may be considered to be stable if the change is within a threshold value of the previous current data (for instance, ±3%). The output of step 725 is a value for the change in normalised current, at step 726. This may be set to zero if the current data is considered to be stable. That is, a positive change may be considered to be a delta larger than a threshold, for instance +3%, similarly for a negative change, and with a delta less than the threshold set to zero. Additionally, in some examples, noise filtering may be performed (for instance, a value of 10 V may be discarded if other BES data is around mV range). In some examples the use of a threshold around 0 for stable reduces the number of alerts. In a further example, it may be that set number of positive (or negative) results in a row may be required before the output 726, 728 is set to indicate a change. For example, a comparison at step 725, 727 may be made every 60 seconds and when 15 positives/negatives/stables in a row are detected then the status would be classed as changed. Steps 725 and 726 are repeated for each subsequent BES sensor stage (the example for the n^{th} stage is shown—steps 727 and 728).

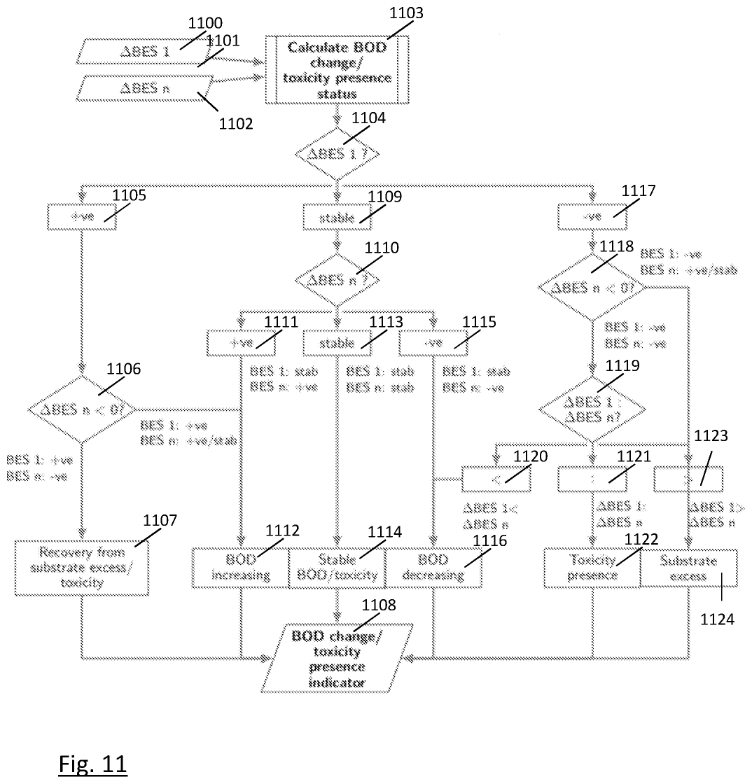

The data indicating the change in normalised BES sensor current for each stage forms an input to step 729, where a determination is made whether the changing current data is indicative of a change in organic loading or a change in toxicity. Step 729 is expanded upon in the description below for the sub routine of FIG. 11. The output of step 729 is one of the following BOD change/toxicity presence indicator statuses: recovery from inhibition (substrate excess/toxicity)—step 730; BOD increasing—step 731; stable BOD/toxicity—step 732; BOD decreasing—step 733; substrate excess—step 734; or toxicity presence—step 730. The respective status provides an input 736 to process monitoring 737 and process control 738.

Process monitoring step 737 comprises receives information from predicted organic load 724 and BOD/toxicity status indicator 736. Near real-time monitoring of processes water quality (organic load and toxicity presence) is enabled. As noted previously, the data may be considered to be near real-time due to the time taken for water to flow through the multi-stage BES sensors. This may be of the order of several minutes to hours (though compares favourably to off-line tests taking days). This may comprise, for instance, an output to a user, for instance displaying organic load and toxicity through a web interface, an app or on-site process monitor display. In some examples an alert may be sent to a user, for instance periodically or when a threshold organic loading is reached, or when the presence of a toxic compound is detected. Methods of data presentation will be familiar to the skilled person but fall outside of the scope of the present invention. Furthermore, process monitoring 737 may comprise recording data to form a historical record for optimisation, compliance, or failure tracking purposes.

Process control 738 comprises the use of the predicted organic load and toxicity data to control process functions. For the example of a municipal waste water treatment plant, by testing influent, effluent or flow between processes, the various processes within the plant may be optimised. The predicted organic load (and optionally toxicity data) may be used within a process control feedback loop in order to maintain organic loading within a predetermined tolerance. This may permit for more efficient operation, while reducing the risk of a pollution event. For instance, aeration rate in an activated sludge process may be controlled if a BES device according to the present invention was installed upstream (that is, post-primary settlement) or in-situ (inside activated sludge tank) in a waste water treatment plant. Process control step 738 may comprise the fully automated control of treatment processes using the data generated according to the flow chart of FIG. 7. Manual intervention may be reduced, providing for further efficiency savings. In some examples, thresholds (either regulatory values or process-defined optimised values) may be used to raise alarms to ensure continued operation of the treatment plant within optimum boundaries. The BOD/toxicity indicator may be used to divert, alter or alert to process changes which may require operator intervention or further sampling for off-line analysis and/or reporting to regulator/downstream users.

While particular examples discussed above relate to waste water quality monitoring, it will be appreciated that this is only one example. Water quality monitoring is desirable or a regulatory requirement in a range of industries and environmental applications. The present invention is not limited to any particular use case scenario. Further examples of applications of the present invention include industrial treatment plant influent, process and effluents, for instance distilleries, breweries, livestock waste, palm oil production, food processing, oilfield and refineries, soap and detergent manufacture, pesticide manufacture, seafood processing, dairy processing, bakeries, soft drink manufacture, textiles, meat processing, rubber processing, potato processing, landfill leachate, pulp and paper mills, olive oil manufacture and the pharmaceutical industry. Additionally, environmental monitoring of water bodies away from known potential pollution sources may also be desirable.

Turning now to FIG. 8, the operation of a BES sensor based upon an MFC configuration will now be described in greater detail. Step 800 (water flow containing organic compounds) corresponds to step 701 of FIG. 7, and step 801 corresponds to one of the BES sensor stages 713 to 717 of FIG. 7. Step 802 comprises the effluent flow to a subsequent BES sensor stage, or out from the BES device.

Water to be analysed comes into contact with an anode electrode biofilm 803 upon an anode electrode 807. As noted previously, an MFC fuel cell forming a BES sensor comprises an external resistor 808 electrically connected between the anode 807 and cathode electrode 809. Accordingly, the MFC fuel cell generates electricity.

The anode electrode 807 (for instance, carbon cloth) provides a base upon which an electrogenic biofilm 803 is grown with bacteria. The bacteria may be enriched directly from the water flowing into the BES sensor (for instance, from municipal sewage) or pre-enriched with a pre-established community (which may be necessary with some abiotic industrial waste waters and environmental applications of the present invention). The anodic biofilm is capable of oxidising biodegradable organic compounds (for instance, acetate) to biodegradation products (for instance, CO₂ 804 and H⁺ 805), resulting in electrons (e⁻) being donated to the anode electrode 807.

Typically the anode and cathode are separated by a separator such as an ion exchange membrane 806, but sometimes no separator is used and they share the same chamber. Membrane 806 may permit certain ions (for instance, H⁺) to pass from the anode to cathode side but prevent bulk mixing for instance, waste water with cathode medium or biofouling. The membrane 806 may also prevent oxygen permeation to enable the anode side to stay anoxic and the cathode side oxic.

The external resistance (R_{Ext}) 808 is electrically connected between the anode 807 and cathode 809. A voltage measurement 813 across the resistance 808 allows the current output to be calculated at step 814. At cathode electrode 809, potentially being coated with a catalyst 810, oxygen 812 is reduced producing water 811. Electrical current flows through the circuit driven by the consumption of organic matter at the anode 807 and oxygen reduction at the cathode 809. The resistance used can alter the detection range and stability of the BES sensor.

However, an MFC is not the only suitable type of fuel cell to form a sensor. Others including a microbial electrolysis cell, a microbial desalination cell and a general bioelectrochemical system cell will be familiar to the skilled person. Turning to FIG. 9, this flow chart illustrates the sub-routine for a Microbial Electrolysis Cell (MEC) fuel cell based sensor according to an example of the present invention. Step 900, 901, 902 correspond to steps 800, 801, 802 of FIG. 8. However, an MEC fuel cell differs in that a power supply 909 is connected between working and counter electrodes 908, 910, providing an extra applied potential driving the electrochemical reactions. A potentiostat may be used instead of a power supply 909, with the further inclusion of a reference electrode 907. That is, the potentiostat 909 regulates the anodic potential of the working electrode 908 in comparison to the reference electrode 907 inserted in the same chamber as the working electrode. As for the MFC fuel cell of FIG. 8, an anodic biofilm 903 is formed on the working electrode 908, which consumes organic matter to produce carbon dioxide 904, hydrogen ions 908 and electrons conducted to the counter electrode 910. In place of the reduction of oxygen in an MFC fuel cell, in an MEC fuel cell the hydrogen ions and free electrons combine at the counter electrode to produce Hydrogen. The current flowing through the potentiometer 909 comprises the output 912.

Further alternative fuel cell arrangements will be well known to the skilled person.

Referring now to FIG. 10, a sub-routine for normalising current from a BES fuel cell current output by a stored value will now be described, corresponding to steps 715, 719 of FIG. 7. This normalisation accounts for differences in electrochemical performance between multi-stage cells. It ensures that each stage is equally weighted for the calculation of current sum at step 721.

The measured BES current 1005 (in Amps) is taken as an input and at step 1006 is used as the numerator and divided through by a normalisation parameter as the denominator. The normalisation parameter corrects for differences in electrochemical performance, both in time as components (such as membrane/cathode begin to foul/degrade), and additionally between different BES fuel cells across the stages. The normalisation parameter may comprise one of:
Maximum current 1000 (measured periodically under saturated organic load conditions);
Open circuit potential (OCP) 1001 (measured periodically with the external resistance/applied potential of the fuel cell disconnected);
Maximum current 1002 (measured periodically under rapid flow-rate conditions);
Maximum power 1003 (measured periodically by polarisation); or
Maximum current 1004 (measured periodically by polarisation curve or potentiostat).

To account for declines in performance over time (for instance, membrane/cathode fouling/degradation) normalisation parameters may be redetermined on a regular basis (for instance, daily, weekly or monthly). In some cases (for instance, maximum current under a rapid flow-rate, or OCP) an automated routine can be incorporated into the device itself. Others (for instance, maximum current under saturated organic load) may require manual interventions or more sophisticated automation, for instance.

Referring now to FIG. 11, a sub-routine to differentiate between BOD change and toxicity presence status (step 729 of FIG. 7) will now be described. Step 1103 initiating the differentiation procedure takes as its input the change in BES current compared to past values for each stage (steps 1101 and 1102, corresponding to steps 726 and 728 of FIG. 7).

The sub-routine of FIG. 11 comprises a decision matrix which begins at step 1104 with a determination whether for the first stage the changes are positive 1105 (current increasing), negative 1117 (current decreasing) or stable 1109 (change is zero or within a threshold range, for instance 3%).

If the change for the first stage is positive (1105) then at step 1106 it is determined whether the change for the last, n^{th} stage is less than zero. If the change for the first stage is positive and the change for the last stage is negative then it is determined that the BES device is in recovery from inhibition (substrate excess/toxicity): step 1107, which is passed as the output indicator at step 1108 (corresponding to step 736 of FIG. 7). If the first stage change is positive and it is determined at step 1106 that the n^{th} stage change is positive or stable then it is determined at step 1112 that BOD is increasing.

If it is determined that the first stage change is stable (1109) then at step 1110 it is determined whether the n^{th} stage change is positive (1111—leading to a determination of increasing BOD 1112); whether the n^{th} stage change is stable (1113—leading to a determination of stable BOD and toxicity 1114); or whether the n^{th} stage change is negative (1115—leading to a determination of decreasing BOD 1116).

If the change for the first stage is negative (1117) then at step 1118 it is determined whether the change for the last, n^{th} stage is less than zero. If the change for the first stage is negative and change for the n^{th} stage is negative then at step 1119 then the magnitude of the absolute change for the first stage compared to the n^{th} stage is determined. If the change for the first stage is smaller (1120) then this indicates that BOD is decreasing (1116). If the change for the first stage is the same as for the n^{th} stage (1121) then this indicates that toxicity is present (1122). If the change for the first stage is larger (1123) then this indicates that there is excess organic substrate (1124).

If the first stage change is negative (1117) and it is determined at step 1118 that the n^{th} stage change is positive or stable then this indicates that there is excess organic substrate (1124).

Referring now to FIG. 14, this is a schematic illustration of a BES device viewed from above according to an example of the present invention. The apparatus principally comprises electronics enclosure 1400, environmental sensor enclosure 1401 and BES enclosure 1402. It will be understood that this arrangement is exemplary and for instance the environmental sensor enclosure 1401 and the BES enclosure 1402 may be combined. The electronics enclosure 1400 includes a data acquisition device 1403 configured to receive data from the environmental sensor enclosure 1401 and the BES enclosure 1402. Data acquisition device 1403 may further perform the processing described above in connection with FIG. 7, and interface with further process monitoring or process control equipment, or the data from the environmental and BES sensors may be output for analysis elsewhere. The environmental sensor enclosure 1401 may include one or more environmental sensors from a group including pH, temperature, conductivity and dissolved oxygen probes 1404. The probes 1404 may be arranged inline to sense water environmental parameters as it flows through. A heater (not illustrated) may be included to maintain the probes at an set temperature. The BES enclosure 1402 includes one or more, for instance three triplicate feed lines 1405 for redundancy, each containing at least one, for instance four BES sensors 1406, arranged hydraulically in series. Furthermore, for temperature regulation, each feed line 1405 may include a heated hose or heated mat 1407, a temperature sensor 1408 and a pressure probe 1409. The pressure probe 1409 may be used to detect blockages, and may not be required for calibration (though it could be used for calibration in some examples of the invention). This configuration is only one example. This serves to bring the water to a desired temperature. In one example the temperature regulation comprises a coil at the inlet of the tubular enclosure providing a stable water temperature (+/−2° C.). No preheating or temperature control of the environmental sensor enclosure is required as these sensors serve to predict the baseline level of biofilm activity (to rule out false negatives/positives for instance, where conditions are not optimal for bacterial activity). Data from all sensors may be collated in the electronics enclosure 1400.

To avoid gas build up when filling or for removing blockages in some examples the BES device may be coupled to an actuator configured to tilt the BES device upwards or downwards. This tilting movement may be in line with the longitudinal axis (that is, the flow direction) of the BES device. In one example the BES device may provided on a platform 1410 which can be tilted (by action of a linear actuator leg not illustrated) upwards to allow gas egress or downwards to drive out biomass accumulated under forced flow. It will be understood that any conventional form of actuator may be used. This tilting movement is not required for all examples.

Close to the inlet of each line 1405 in the BES enclosure and the environmental sensor enclosure will be a pumphead and pump (not illustrated) such as a peristaltic pump which continuously pumps water to be analysed into the hydraulic system.

Referring now to FIG. 15, this is a flow chart illustrating water quality monitoring algorithm according to a further example of the present invention. The skilled person will appreciate that the detail of each step is presented above in relation to the corresponding part of FIG. 7. The method starts at step 1500 and at step 1501 a water sample to be processed is received. This is fed to a BES sensor, for instance a BES fuel cell, at step 1502. This generates BES sensor data at step 1503 which is fed to an organic loading prediction algorithm at step 1505 along with other parameter data 1504. The other parameter data 1504 may comprise one or more of at least one environmental parameter, at least one piece of configuration data for the BES sensor or at least one parameter for a system in which the BES sensor is implemented. The output from the algorithm at step 1505 is a parameter indicative of organic compound concentration for the water sample. Optionally, the method may end here. Or optionally, it may be passed to a step of process monitoring 1507 or a step of process control 1508. The method ends at step 1509.

It will be appreciated that examples of the present invention can be realized in the form of hardware, software or a combination of hardware and software. Any such software may be stored in the form of volatile or non-volatile storage, for example a storage device like a ROM, whether erasable or rewritable or not, or in the form of memory, for example RAM, memory chips, device or integrated circuits or on an optically or magnetically readable medium, for example a CD, DVD, magnetic disk or magnetic tape or the like. It will be appreciated that the storage devices and storage media are examples of machine-readable storage that are suitable for storing a program or programs comprising instructions that, when executed, implement examples of the present invention.

Accordingly, examples provide a program comprising code for implementing apparatus or a method as claimed in any one of the claims of this specification and a machine-readable storage storing such a program. Still further, such programs may be conveyed electronically via any medium, for example a communication signal carried over a wired or wireless connection and examples suitably encompass the same.

Throughout this specification, the words “comprise” and “contain” and variations of them mean “including but not limited to”, and they are not intended to (and do not) exclude other components, integers or steps. Throughout this specification, the singular encompasses the plural unless the context otherwise requires. In particular, where the indefinite article is used, the specification is to be understood as contemplating plurality as well as singularity, unless the context requires otherwise. Throughout this specification, the term “about” is used to provide flexibility to a range endpoint by providing that a given value may be “a little above” or “a little below” the endpoint. The degree of flexibility of this term can be dictated by the particular variable and can be determined based on experience and the associated description herein.

Features, integers or characteristics described in conjunction with a particular aspect or example of the invention are to be understood to be applicable to any other aspect or example described herein unless incompatible therewith. All of the features disclosed in this specification, and/or all of the steps of any method or process so disclosed, may be combined in any combination, except combinations where at least some of such features and/or steps are mutually exclusive. The invention is not restricted to the details of any foregoing examples. The invention extends to any novel feature or combination of features disclosed in this specification. It will be also be appreciated that, throughout this specification, language in the general form of “X for Y” (where Y is some action, activity or step and X is some means for carrying out that action, activity or step) encompasses means X adapted or arranged specifically, but not exclusively, to do Y.

Each feature disclosed in this specification may be replaced by alternative features serving the same, equivalent or similar purpose, unless expressly stated otherwise. Thus, unless expressly stated otherwise, each feature disclosed is one example only of a generic series of equivalent or similar features.

The reader's attention is directed to all papers and documents which are filed concurrently with or previous to this specification in connection with this application and which are open to public inspection with this specification, and the contents of all such papers and documents are incorporated herein by reference.

1. A water quality monitoring method, the method comprising:
receiving BioElectrochemical System, BES, sensor data indicating an output from at least one BES sensor exposed to a water sample;
receiving data indicating at least one environmental parameter, at least one piece of configuration data for the BES sensor or at least one parameter for a system in which the BES sensor is implemented; and
processing the received data according to a calibration algorithm to generate a parameter indicative of organic compound concentration for the water sample.

2. A method according to claim 1, wherein the BES sensor comprises a Microbial Fuel Cell, MFC, comprising an electrode coated with electrogenic bacteria arranged such that as the water sample flows past the electrode, organic matter is consumed by the electrogenic bacteria to generate an electrical current, the BES sensor output being indicative of that electrical current.

3. A method according to claim 1, wherein the BES sensor data is indicative of the sum of BES sensor outputs from a plurality of BES sensors arranged hydraulically in series such that the water sample flows through the plural BES sensors or the outputs of each BES sensor individually.

4. A method according to claim 3, wherein for a plurality of BES sensors the BES sensor data is indicative of normalised current data for each BES sensor.

5. A method according to claim 1, wherein the at least one environmental parameter data comprises data indicative of one or more of:
the temperature of the water sample;
the temperature of the ambient air;
the electrical conductivity of the water sample;
the pH of the water sample;
the dissolved oxygen level of the water sample; or
the rainfall recorded in proximity of the sensor;
wherein the at least one piece of configuration data for the BES sensor comprises data indicative of one or more of:
external resistance of a BES sensor;
electrode size;
electrode spacing;
chamber volume;
membrane type;
catalyst type;
biofilm age;
maximum power;
maximum current;
open circuit potential; and
wherein the at least one parameter for a system in which the BES sensor is implemented comprises data indicative of one or more of:
water flow rate; or
water tank level.

6. A method according to claim 1, wherein the calibration algorithm comprises an AI algorithm, machine learning algorithm or multiple regression trained according to a data set comprising, for a plurality of water samples, BES sensor data, validation data indicating organic compound concentration for the water samples determined according to an alternative test, and at least one of environmental parameter data or BES sensor configuration data.

7. A method according to claim 6, wherein the alternative test comprises at least one of:
Biological Oxygen Demand, BOD;
Chemical Oxygen Demand, COD; or
Total Organic Carbon, TOC.

8. A method according to claim 3, further comprising:
determining a change in BES sensor data between a first time point and a second time point for each of the plurality of BES sensors; and
determining, based on the change for each BES sensor, an indication whether the change is indicative of a change in organic compound concentration, the presence of toxicity, excessive organic compound concentration or sensor recovery from excessive organic compound concentration or toxicity.

9. A method according to claim 1, further comprising:
monitoring organic compound concentration based on the parameter indicative of organic compound concentration; or
controlling process flows in a waste water treatment plant or industrial process based on the parameter indicative of organic compound concentration in order to maintain organic compound concentration within a predetermined tolerance.

10. A method according to claim 9, wherein monitoring organic compound concentration or controlling process flows is further based on the indication whether the change is indicative of a change in organic compound concentration, the presence of toxicity, excessive organic compound concentration or sensor recovery from excessive organic compound concentration or toxicity.

11. A computer-readable storage medium having computer-readable program code stored therein that, in response to execution by a processor, cause the processor to perform the method of claim 1.

12. An apparatus comprising a processor and a memory storing executable instructions that, in response to execution by the processor, cause the apparatus to perform the method of claim 1.

13. A water quality monitoring device comprising:
a BES sensor configured to generate a current output when exposed to a water sample; and
a processor configured to receive data from the BES sensor and data indicating at least one environmental parameter, at least one piece of configuration data for the BES sensor or at least one parameter for a system in which the BES sensor is implemented and to execute a calibration algorithm to generate a parameter indicative of organic compound concentration.

14. A device according to claim 13, wherein the processor is further configured to perform the method of claim 2.

15. A device according to claim 13, further comprising an actuator coupled to the BES sensor configured to selectively tilt the biosensor upwards or downwards.

16. A device according to claim 1, further comprising a plurality of BES sensors arranged hydraulically in series such that the water sample flows through the plural BES sensors, each BES sensor being configured to generate a current output when exposed to a water sample.
